(19) United States
(12) Patent Application Publication
(10) Pub. No.: US 2015/0157216 A1
(43) Pub. Date: Jun. 11, 2015

(54) DEVICE, SYSTEM, AND METHOD FOR ASSESSING INTRAVASCULAR PRESSURE

(71) Applicant: Volcano Corporation, San Diego, CA (US)

(72) Inventors: Jeremy Stigall, Carlsbad, CA (US); Joseph Lauinger, San Diego, CA (US)

(21) Appl. No.: 14/562,388

(22) Filed: Dec. 5, 2014

Related U.S. Application Data
(60) Provisional application No. 61/913,065, filed on Dec. 6, 2013.

(51) Int. Cl. A61B 5/0215; A61B 5/00; A61B 5/02

(52) U.S. Cl. CPC A61B 5/0215; A61B 5/02007; A61B 5/7278

(57) ABSTRACT

What is described is an apparatus for intravascular pressure measurement, comprising an elongate body and a first pressure sensor. The elongate body includes a proximal portion and a distal portion, the body defines a distal guidewire lumen extending adjacent a sensor assembly along a distal portion of the system. The first pressure sensor is disposed within the wall of the distal portion of the body. An exterior surface of the sensor and the outer surface of the body are substantially aligned.

### CROSS REFERENCE TO RELATED APPLICATION

The present application claims priority to and the benefit of U.S. Provisional Patent Application No. 61/913,065 filed Dec. 6, 2013, which is hereby incorporated by reference herein in its entirety.

### TECHNICAL FIELD

Embodiments of the present disclosure relate generally to the field of medical devices and, more particularly, to a device, system, and method for assessing pressure within vessels. In particular, the present disclosure relates to the assessment of the severity of a blockage or other restriction to the flow of fluid through a vessel. Aspects of the present disclosure are particularly suited for evaluation of biological vessels in some instances. For example, some particular embodiments of the present disclosure are specifically configured for the evaluation of a stenosis of a human blood vessel.

### BACKGROUND

Heart disease is a serious health condition affecting millions of people worldwide. One major cause of heart disease is the presence of blockages or lesions within the blood vessels that reduce blood flow through the vessels. Traditionally, surgeons have relied on X-ray fluoroscopic (planar) images to show the external shape or silhouette of the blood vessels to guide treatment. Unfortunately, using only X-ray fluoroscopic images lends a great deal of uncertainty about the exact extent and orientation of the lesion responsible for the occlusion, making it difficult to find the exact location of the stenosis for treatment. In addition, X-ray fluoroscopy is an inadequate reassessment tool to evaluate the vessel after surgical treatment.

A currently accepted technique for assessing the severity of a stenosis in a blood vessel, including ischemia-causing lesions, is fractional flow reserve (FFR). FFR is defined as the ratio of the maximal blood flow in a stenotic artery, taken distal to the lesion, to normal maximal flow. Accordingly, to calculate the FFR for a given stenosis, two blood pressure measurements are taken: one measurement distal or downstream to the stenosis and one measurement proximal or upstream to the stenosis. FFR is a calculation of the ratio of the distal pressure measurement relative to the proximal pressure measurement. FFR provides an index of stenosis severity that allows determination as to whether the blockage limits blood flow within the vessel to an extent that treatment is required. The more restrictive the stenosis, the greater the pressure drop across the stenosis, and the lower the resulting FFR. FFR measurements can be used as a decision point for guiding treatment decisions. The normal value of FFR in a healthy vessel is 1.00, while values less than about 0.80 are generally deemed significant and require treatment. Common treatment options include angioplasty, atherectomy, and stenting.

One method of measuring the pressure gradient across a lesion is to use a pressure sensing guidewire that has a pressure sensor embedded within the guidewire itself. A user may initially position the pressure sensor of the guidewire distal to the lesion and measure the distal pressure before drawing the guidewire backwards to reposition the sensor proximal to the lesion to measure the proximal pressure. This method has the disadvantages of inaccurate pressure readings due to drift and increased susceptibility to thermal variations, difficulty maneuvering a guidewire through blockages, high manufacturing costs, and time-consuming repositioning steps (especially in situations involving multiple lesions). Further, pressure-sensing guidewires often suffer from reduced precision and accuracy in making intravascular pressure measurements when compared to larger pressure-sensing devices, such as aortic pressure-sensing catheters.

Another method of measuring the pressure gradient across a lesion is to use a small catheter connected to a blood pressure sensor, which is often contained in a sensor housing associated with the catheter. However, this method can introduce error into the FFR measurement because as the catheter crosses the lesion, the catheter and the sensor housing themselves create additional blockage to blood flow across the lesion and contributes to a lower distal blood pressure than what would be caused by the lesion alone, which may exaggerate the measured pressure gradient across the lesion.

While the existing treatments have been generally adequate for their intended purposes, they have not been entirely satisfactory in all respects. The devices, systems, and associated methods of the present disclosure overcome one or more of the shortcomings of the prior art.

### SUMMARY

The present disclosure relates generally to a device, systems, and methods of using a pressure-sensing catheter for the assessment of intravascular pressure, including, by way of non-limiting example, the calculation of a FFR value. In some instances, embodiments of the present disclosure are configured to measure the pressure proximal to and distal to a stenotic lesion within a blood vessel. Embodiments of the present disclosure include a pressure sensor embedded in the wall of the catheter or have a movable sleeve capable of smoothing the outer diameter of the sensing catheter. In some embodiments, the pressure-sensing catheter disclosed herein is configured as a monorail or rapid exchange catheter where the guidewire exits the catheter body adjacent the distal end. In other embodiments, the pressure-sensing catheter disclosed herein is configured as a conventional over-the-wire catheter. The pressure-sensing catheters disclosed herein enable the user to obtain pressure measurements using an existing guidewire (e.g., a conventional 0.014 inch guidewire) that can remain fairly stationary through the pressure measurement procedure. Thus, the pressure-sensing catheters disclosed herein enable the user to obtain physiologic information about an intravascular lesion upon pullback of the catheter without losing the original position of the guidewire.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a diagrammatic partial perspective view of a sensing system according to one embodiment of the invention.

FIG. 2A is a diagrammatic partial perspective view of a sensing system according to a second embodiment of the invention

FIG. 2B illustrates a perspective view of an alternative sensing catheter similar to FIG. 2A.

FIG. 3 is a partial perspective view of a further embodiment of a sensing catheter according to another aspect of the present invention.

FIG. 4 is partial perspective view of still a further embodiment of a sensing catheter according to another aspect of the present invention.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. For the sake of brevity, however, the numerous iterations of these combinations will not be described separately. For simplicity, in some instances the same reference numbers are used throughout the drawings to refer to the same or like parts.

The present disclosure relates generally to a device, systems, and methods of using a pressure-sensing catheter for the assessment of intravascular pressure, including, by way of non-limiting example, the calculation of a FFR value. In some instances, embodiments of the present disclosure are configured to measure the pressure proximal to and distal to a stenotic lesion within a blood vessel. Embodiments of the present disclosure include a pressure sensor embedded in the wall of the catheter or have a movable sleeve capable of smoothing the outer diameter of the sensing catheter. In some embodiments, the pressure-sensing catheter disclosed herein is configured as a monorail or rapid exchange catheter where the guidewire exits the catheter body adjacent the distal end. In other embodiments, the pressure-sensing catheter disclosed herein is configured as a conventional over-the-wire catheter. The pressure-sensing catheters disclosed herein enable the user to obtain pressure measurements using an existing guidewire (e.g., a conventional 0.014 inch guidewire) that can remain fairly stationary through the pressure measurement procedure. Thus, the pressure-sensing catheters disclosed herein enable the user to obtain physiologic information about an intravascular lesion upon pullback of the catheter without losing the original position of the guidewire.

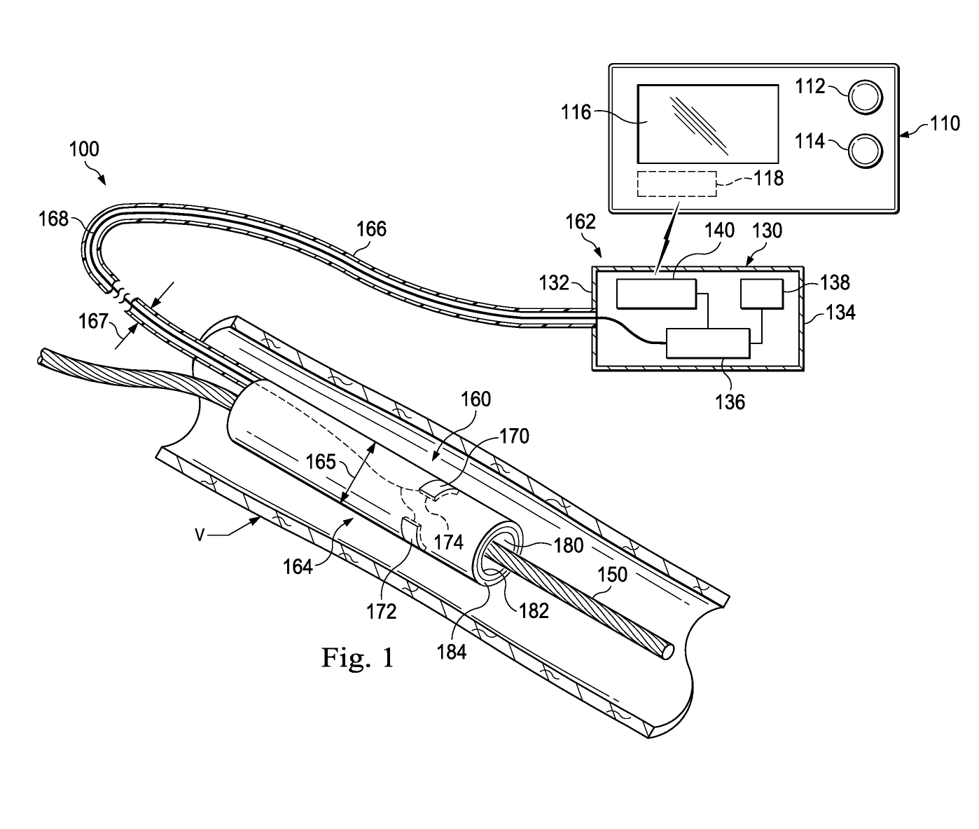

FIG. 1 illustrates a medical system 100 that is configured to measure pressure within a tubular structure V (e.g., a blood vessel) according to one embodiment of the present disclosure. In some embodiments, the medical system 100 is configured to calculate FFR based on the obtained pressure measurements. The system 100 includes a pressure-sensing catheter 160 interconnected with a processing and communication system 130 that is communicatively coupled to a user interface 110.

FIG. 1 illustrates a pressure sensor 170 embedded in the catheter wall 184. Both for this embodiment and the other embodiments disclosed herein, the pressure sensor 170 comprises any type of pressure sensor that is sufficiently stress resistant to maintain functionality while embedded within the catheter wall 184. For example, the pressure sensor 170 may comprise a capacitive sensor, a piezoresistive pressure transducer, a fiber optic pressure sensor such as disclosed in U.S. Pat. Nos. 8,298,156 and 8,485,985 and Application Nos. 2013/0303914 and 2013/0131523 (each incorporated by reference herein in their entirety), a sensor with a silicon backbone, or any other type of pressure sensor having the requisite durability and stress resistance. In some instances, the sensor 170 includes an array or plurality of sensor elements (e.g., a capacitive pressure sensor array). In the pictured embodiment, the sensor 170 includes a sensor diaphragm assembly. In some embodiments, the sensor diaphragm assembly includes a body having a recess covered by a flexible diaphragm configured to measure fluid pressure. The diaphragm may flex in response to variations in pressure around the diaphragm, thereby reflecting variations in blood pressure, for example. The sensor 170 can then measure and transmit the variations in pressure imparted on the diaphragm assembly. Still further, although the illustrated catheter is described in terms of a pressure sensor, it is contemplated that the type of sensing element disposed on the catheter is not a limitation with respect to all of the teachings of the present disclosure. More specifically, it is contemplated that while one sensor on the catheter may be a pressure sensor, an additional one or more imaging or flow sensors can be incorporated as the sensors of the present disclosure. For example, the sensors 170 and 172 could be ultrasonic transducers that can image the surrounding vessel such as by intravascular ultrasound (IVUS) and/or detect fluid flow in the vessel.

In the pictured embodiment, the sensor 170 is positioned within a sensor recess defined within the catheter wall. In some embodiments, the sensor 170 is in intimate contact with the wall. The sensor may be coupled to the catheter wall using any of a variety of known connection methods, including by way of non-limiting example, welding, biologically-compatible adhesive, and/or mechanical fasteners. For example, in one embodiment, the sensor is adhesively bonded to the sensor recess using Loctite 3311 or any other biologically compatible adhesive. In some embodiments, the sensors may be integrally formed with the catheter wall. In some embodiments, the sensor recess may be radiopaque.

In one aspect, the sensing catheter 160 is coupled to the processing and communication system 130 such that elongated catheter extension body 166 engages housing 134 while conductors 168 are electrically coupled to application specific integrated circuit (ASIC) 136. Battery 138 powers ASIC 136 and transmitter 140 that communicates wirelessly with user interface 110 in a standard format such as WiFi or Bluetooth. ASIC 136 can provide energizing signals to the sensors 170 and 172 along conductors 168. In one aspect, sensors 170 and 172 are resistive pressure sensors and ASIC 136 receives analog signals from the sensors, processes the signals and provides digital signals to transmitter 140. In one aspect, the proximal end 162 of the catheter 160 is integrally formed with the processing and communication system 130 such that end wall 132 extends directly from catheter body 166 into an enlarged housing area 134 containing the power, processing and communication features of the system. It will be appreciated, that the catheter, including the communication and processing system 130 can be have a unitary, water tight outer surface surrounding the components and that the entire system can be made as a single use disposable item.

User interface 110 includes a wireless communication receiver 118 configured to receive signals from the transmitter 140. The user interface includes processing components (not shown) that can control the display 116 and receive user inputs from buttons 112 and 114. In one form, the raw data provided by the catheter system is displayed directly on display 116. In another form, the user interface 110 is controlled by a user through inputs 112 and 114 to collect sufficient data to calculate a fractional flow reserve (FFR) for a vessel and display that information to the user.

The catheter 160 includes an intermediate portion 166 comprising an elongate, flexible, tubular body extending from the proximal portion 162 to the distal sensing portion 164. The body 166 comprises a catheter wall that defines an internal lumen configured to receive conductors 168. In the illustrated embodiment, intermediate portion 166 has a smaller diameter than the diameter 167 than the diameter 165 of the distal portion 164. The distal portion 164 includes two embedded sensors 170 and 172. Although two sensors are shown for the purposes of illustration, in some applications only a single sensor is needed, while in other applications a plurality of sensors may be needed. The exterior of sensors 170 and 172 is not greater than the diameter 165 of the distal portion 164 and is generally flush with the outer surface.

The distal portion 164 is constructed by first providing an inner catheter portion 182 defining a guidewire lumen 180 configured to receive a guidewire 150. Sensors 170 and 172 are mounted on the exterior of the inner catheter 182 and electrically connected to the conductors 168. Outer catheter portion 184 is then positioned to surround the inner catheter 182. Outer catheter includes a pair of openings sized to receive the sensors 170 and 172. In one form, the outer catheter 184 is positioned with the openings adjacent the sensors and then heat shrunk to match the outer diameter of the inner catheter 182. In a preferred form, the outer catheter 184 has a material thickness after heat shrinking that is at least as thick as the sensors 170 and 172 extend outwardly from the surface of the inner catheter 182. As a result, as the sensing catheter is advanced along the guidewire within a patient, the sensors will be protected and will not create a protrusion that may catch on patient anatomy.

In general, the catheter 160 is sized and shaped for use within an internal structure of a patient, including but not limited to a patient's arteries, veins, heart chambers, neurovascular structures, gastrointestinal system, pulmonary system, and/or other areas where internal access of patient anatomy is desirable. In the pictured embodiment, the catheter 160 is shaped and sized for intravascular placement.

In particular, the catheter 160 is shaped and configured for insertion into a lumen of a blood vessel V such that a longitudinal axis of the catheter aligns with a longitudinal axis of the vessel at any given position within the vessel lumen. In that regard, the straight configuration illustrated in FIG. 1 is for exemplary purposes only and in no way limits the manner in which the catheter may curve in other instances. Generally, the elongate body may be configured to take on any desired arcuate profile when in the curved configuration. The catheter is formed of a flexible material such as, by way of non-limiting example, plastics, high density polyethylene, polytetrafluoroethylene (PTFE), Nylon, block copolymers of polyamide and polyether (e.g., PEBAX), thermoplastic, polyimide, silicone, elastomers, metals, shape memory alloys, polyolefin, polyether-ester copolymer, polyurethane, polyvinyl chloride, combinations thereof, or any other suitable material for the manufacture of flexible, elongate catheters.

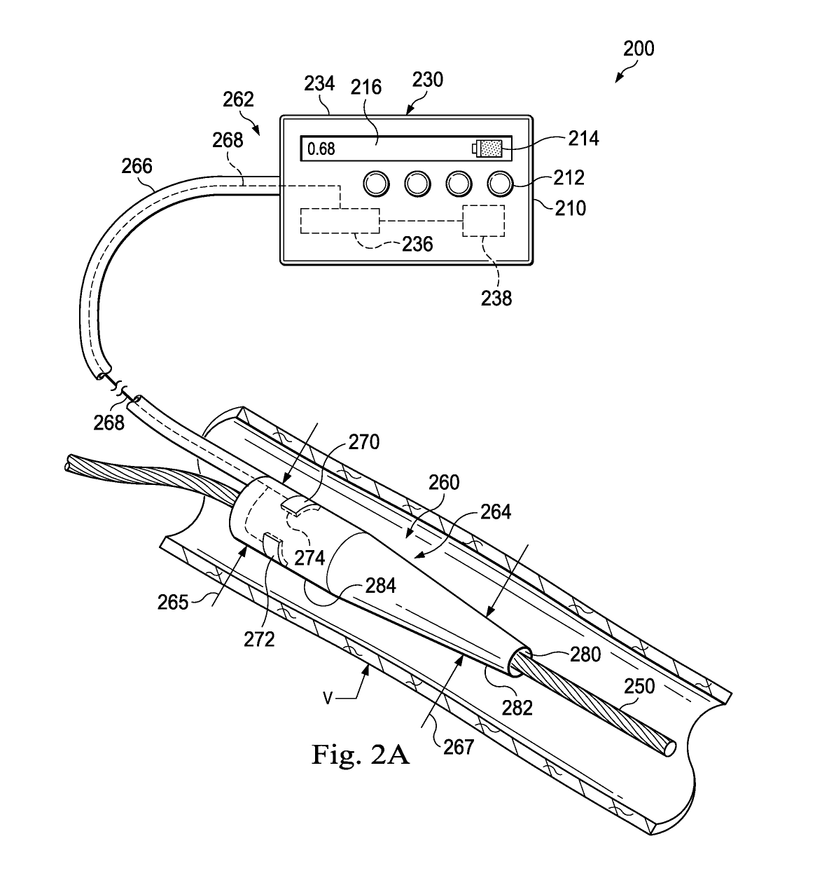

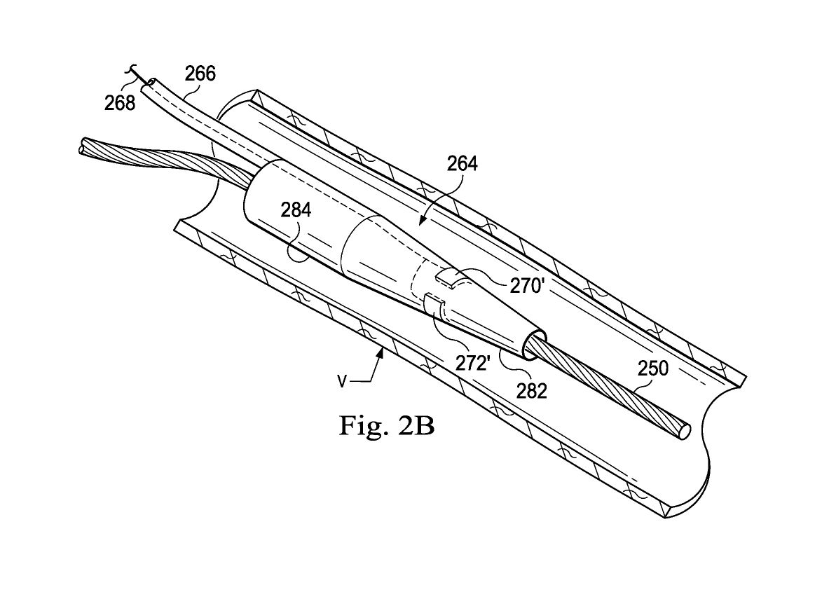

Referring now to FIG. 2A, there is shown still a further embodiment of a sensing catheter according to the present invention. FIG. 2A illustrates a medical system 200 that is configured to measure pressure within a tubular structure V (e.g., a blood vessel) according to one embodiment of the present disclosure. In some embodiments, the medical system 200 is configured to calculate FFR based on the obtained pressure measurements. The system 200 includes a pressure-sensing catheter 260 interconnected with a processing and communication system 230. In one aspect, the sensing catheter 260 is coupled to the processing and communication user interface 230 such that elongated catheter extension body 266 engages housing 234 while conductors 268 are electrically coupled to application specific integrated circuit (ASIC) 236 disposed within housing 234. Battery 238 powers ASIC 136 and display 216. In one form, the housing 234 can also contain a transmitter that communicates wirelessly with a further user interface or other system component in a standard format such as WiFi or Bluetooth. ASIC 136 can provide energizing signals to the sensors 270 and 272 along conductors 268. In one aspect, sensors 270 and 272 are resistive pressure sensors and ASIC 236 receives analog signals from the sensors, processes the signals and provides digital signals the display 216. In one aspect, the proximal portion 262 of the catheter 260 is integrally formed with the processing and communication system user interface 230 such that housing 234 extends directly from catheter body 266 as an enlarged housing area containing the power, processing and communication features of the system. It will be appreciated, that the catheter, including the communication and processing system user interface 230 can be have a unitary, water tight outer surface surrounding the components and that the entire system can be made as a single use disposable item.

The user interface 230 includes an ASIC component 236 that energize on or more sensors 270 and 272, receive signals from the sensors, process the sensor data and output results to the display 216 (and transmit wirelessly if desired). In one form, the raw data provided by the catheter system is displayed directly on display 216. In another form, the user interface 230 is controlled by a user through inputs 112 to collect sufficient data to calculate a fractional flow reserve (FFR) for a vessel and display that information to the user. In one example, when positioning the catheter at a first vessel location, a user input 212 is depressed to take sensor data for a reference pressure. Then at a more distal vessel location, a further button 212 is depressed to take distal pressure. Using the user interface, the processor 236 is then utilized to determine the FFR for the distal vessel location. The results of the FFR are then displayed to the user on the display 216. In the illustrated embodiment, the entire system is powered by a battery 238. In one aspect, the user interface 230 can include an induction coil to permit recharging of the battery.

The catheter 260 includes an intermediate portion 266 comprising an elongate, flexible, tubular body extending from the proximal portion 262 to the distal sensing portion 264. The body 266 comprises a catheter wall that defines an internal lumen configured to receive conductors 268. The distal portion 264 includes two embedded sensors 270 and 272. Although two sensors are shown for the purposes of illustration, in some applications only a single sensor is needed, while in other applications a plurality of sensors may be needed. The exterior of sensors 270 and 272 is not greater than the diameter 265 of the distal portion 164 and is generally flush with the outer surface. The distal section of the distal portion 264 defines outer tapered surface 282 that transitions from a cylindrical section 284 containing the sensors to the distal tip having the guidewire lumen 280 configured to receive the guidewire 250. The tapered outer surface has an outer diameter 267 that is less than the diameter 265.

Referring now to FIG. 2B, the illustrated sensing catheter has the same features as those shown in FIG. 2A, with the exception that sensors 270′ and 272′ are positioned along the tapered surface 282. As illustrated, the sensors are embedded into the catheter such that the sensors do not protrude outwardly from the tapered surface.

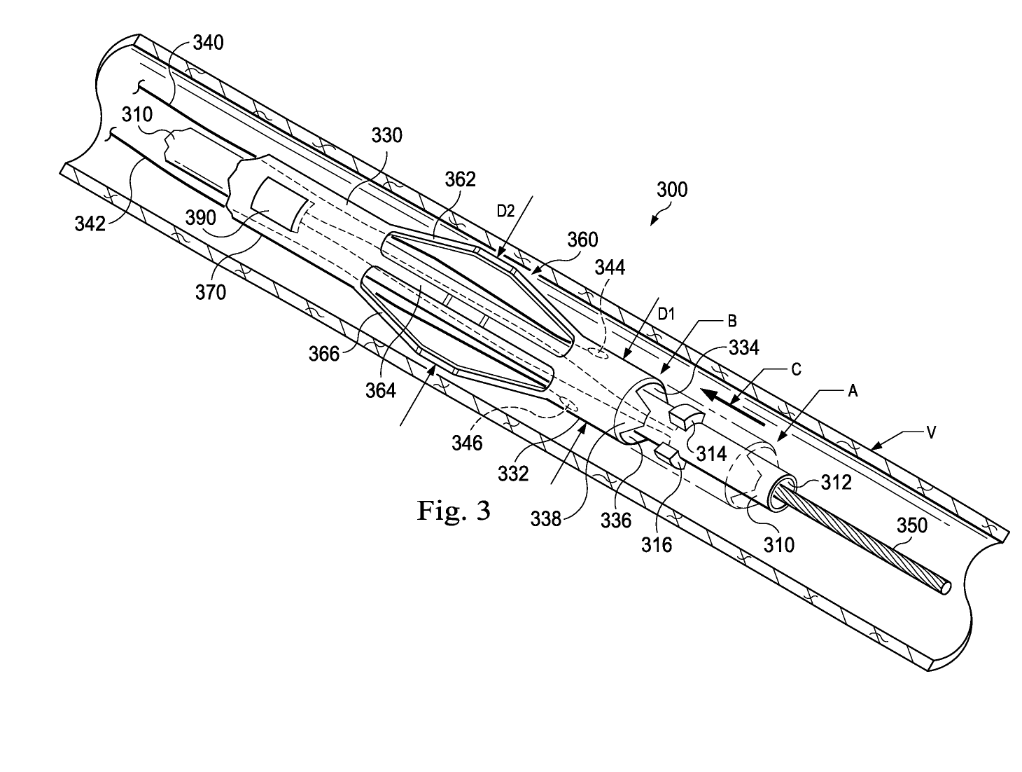

Referring now to FIG. 3, there is shown still a further embodiment of a sensing catheter according to another aspect of the present invention. The sensing catheter 300 includes an inner catheter 310 defining an inner lumen 312 configured to receive a guidewire 350. A pair of sensors 314 and 316 are mounted on the outside of catheter 310 and extend outwardly therefrom. The sensors are electrically connected to ASIC 390 which can process analog signals from the sensor and transmit corresponding data signals to the proximal end of the catheter. An outer catheter 330 is mounted about inner catheter 310. Proximal portion 370 is fixed in relation to inner catheter 310 while distal portion 332 is moveably mounted on the inner catheter. A centering assembly 360 is defined by the outer catheter between the proximal portion 370 and the distal portion 332. In the illustrated embodiment, the centering assembly 360 includes a plurality of elongated deformable legs 362, 364, and 366 defined by cutting elongated apertures in the outer catheter 330. A pair of pull wires 340 and 342 extend through lumens within the outer catheter 330, across the centering assembly and are anchored in the distal portion 332 at anchor points 344 and 346, respectively. The distal portion 332 includes a pair of groove 334 and 336 sized to slidably receive the sensors 314 and 316, respectively, protruding from the inner catheter 310. The entrance to each groove 334 and 336 begins in the tapered end 338.

In operation, the catheter assembly is initially in an insertion configuration with the distal portion 332 extended to the position A with the centering assembly in a collapsed configuration having an outer diameter substantially matching diameter D1. In one aspect, the material of outer catheter 330 has sufficient resiliency such that when no force is applied to the pull wires 340 and 342, the centering assembly is biased to return to the collapsed configuration. The center is advanced over the guidewire 350 to position the sensors in the desired location. When force is applied to the pull wires 340 and 342 in the direction of arrow C, the distal portion 332 slides longitudinally along the inner catheter 310 to the position B to expose the sensors 314 and 316. Since the distal portion 370 of the outer catheter is fixed in relation to the inner catheter the elongated legs 362, 364, and 366 (along with a fourth leg not shown), deform outwardly to the centering configuration illustrated in FIG. 3. In the centering configuration the legs have an outer diameter of D2 that is substantially larger than diameter D1. Thus, as shown, the centering assembly centers the sensors in the middle of the vessel V. The deformable legs are sized to limit their impact on the blood flow in the vessel. In this way, the sensors are positioned in the ideal location within the vessel to obtain the best possible reading of the fluid, including such characteristics as pressure and flow.

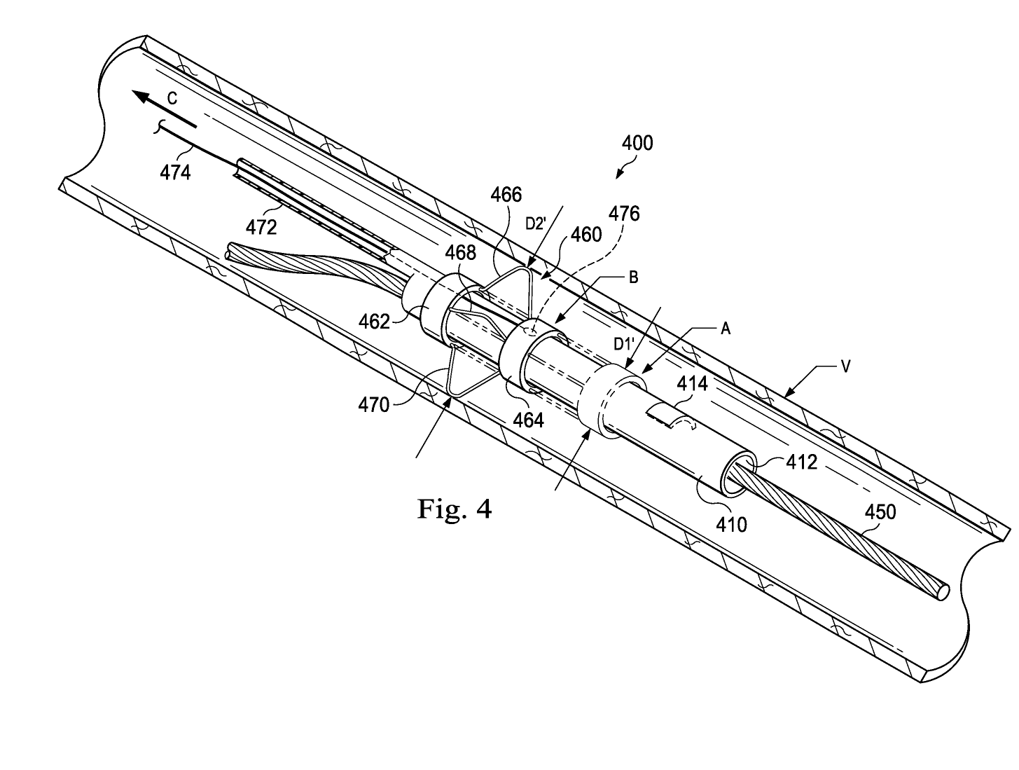

Referring now to FIG. 4, there is shown still a further embodiment of a sensing catheter 400 having a sensor 414 disposed on a distal catheter body 410 defining guidewire lumen 412 that receives guidewire 450. The catheter 400 includes a centering assembly 460 comprising a distal ring 464 slidably mounted on the distal catheter body 410 and a proximal ring 462 fixed to the catheter body 410. A plurality of wires 466, 468 and 470 (other wires on the back side are not shown) interconnect the distal ring and the proximal ring. A pull wire 474 extends through proximal catheter body 472 into the distal catheter body 410 and is connected to distal ring 464 at anchor point 476. Tension applied on pull wire 474 in the direction of arrow C moves distal ring 464 toward proximal ring 462, thereby deforming wires 466, 468, and 470 outwardly to the centering configuration shown in FIG. 4. In the centering configuration with distal ring 464 positioned at position B, the assembly has an outer diameter of D2′ that is much larger than the diameter D1′ of the assembly in the insertion configuration. The wires 466, 468 and 470 are resilient and bias the assembly into the collapsed insertion configuration with distal ring positioned at position A. In the illustrated embodiment, the centering assembly is mounted on the exterior of the catheter 410, however, it is contemplated that in an alternative embodiment, an annular groove may extend around the catheter 410 such that in the collapsed position, the centering assembly has a maximum outer diameter that is less than or the same as the diameter of the catheter 410.

With reference to the above described embodiments, the guidewire lumen includes an internal diameter that is sized and shaped to accommodate the passage of a standard guidewire. The internal diameter may range from 0.014 in. to 0.40 in. In one embodiment, the internal diameter is 0.016 in. to slidingly accommodate a 0.014 in. diameter guidewire. In one embodiment, the internal diameter is 0.024 in. In one embodiment, the internal diameter is 0.018 in. In another embodiment, the internal diameter is 0.038 in. to accommodate a 0.035 in. diameter guidewire. The catheter includes an outer diameter that is sized and shaped to traverse bodily passageways. In the pictured embodiment, the outer diameter is sized to allow passage of the catheter through vascular passageways. In some instances, as mentioned above, the body has an external diameter ranging from 0.014 inches to 0.050 inches. In one embodiment, the outer diameter is 0.024 in. with an internal diameter of about 0.016 in. In one embodiment, the outer diameter is 0.018 in. In another embodiment, the outer diameter is 0.035 in.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. For example, the pressure-sensing catheters disclosed herein may be utilized anywhere with a patient's body, including both arterial and venous vessels, having an indication for pressure measurement. It is understood that such variations may be made to the foregoing without departing from the scope of the present disclosure. Accordingly, it is appropriate that the appended claims be construed broadly and in a manner consistent with the present disclosure.

What is claimed is:

1. A sensing catheter for vessel pressure measurement, comprising:
an elongate catheter body including a proximal portion, an intermediate portion having a first perimeter and a distal portion having a second perimeter greater than the first perimeter, the distal portion defining a lumen extending from a proximal guidewire opening to a distal guidewire opening, the lumen sized and shaped to allow the passage of a guidewire therethrough, the body including an annular wall extending from the lumen to an outer surface of the distal portion; and
a pressure sensor disposed within the annular wall of the body such that an exterior surface of the pressure sensor and the outer surface of the distal portion are substantially aligned to form a smooth outer surface for the distal portion adjacent the pressure sensor.

2. The apparatus of claim 1, wherein the outer surface of the distal portion has a proximal diameter and at least one distal taper have a reduced diameter adjacent the distal portion.

3. The apparatus of claim 2, wherein the pressure sensor is disposed within the distal taper.

4. The apparatus of claim 2, wherein the pressure sensor is disposed within the proximal diameter.

5. The apparatus of claim 1, further including a plurality of pressure sensors disposed within the annular wall of the distal portion of the body.

6. The apparatus of claim 1, further including a centering assembly disposed adjacent the pressure sensor, the centering assembly having a first collapsed position and a second expanded position adapted to engage a vessel wall to center the pressure sensor within the vessel.

7. The apparatus of claim 6, wherein the centering assembly includes at least three outwardly extending legs.

8. The apparatus of claim 6, wherein the centering assembly takes up less than 50% of the volume in the vessel.

9. The apparatus of claim 6, wherein the centering assembly is mounted on the outer surface of the distal portion.

10. The apparatus of claim 6, wherein the centering assembly is recessed within the outer surface of the distal portion when in the collapsed position.

11. The apparatus of claim 1, wherein the outer surface of the body has a diameter of 0.035 inches or less.

12. The apparatus of claim 11, wherein the lumen has an internal diameter of at least 0.014 inches and the sensor is disposed between the lumen and the outer surface.

13. An apparatus for intravascular sensing system, comprising:
an elongate catheter body including a proximal portion, an intermediate portion having a first perimeter and a distal portion having a second perimeter greater than the first perimeter, the distal portion defining a lumen extending from a proximal guidewire opening to a distal guidewire opening, the lumen sized and shaped to allow the passage of a guidewire therethrough, the body including an annular wall extending from the lumen to an outer surface of the distal portion; and
a first sensor joined to the annular wall of the distal portion; and
a sensor cover mounted on the outer surface of the distal portion, the sensor cover movable from a distal sensor covering position to a proximal sensor exposed position spaced proximally from the sensor.

14. The apparatus of claim 13, wherein the sensor cover includes a tapered distal end.

15. The apparatus of claim 13, wherein the sensor cover includes a recess to receive at least a portion of the sensor protruding outwardly from the outer surface of the distal portion.

16. The apparatus of claim 13, further including a centering assembly disposed adjacent the pressure sensor, the centering assembly having a first collapsed position and a second expanded position adapted to engage a vessel wall to center the pressure sensor within a vessel.

17. A method, comprising
advancing a guidewire through a vessel to an area of interest;
positioning a sensing catheter over the guidewire, the sensing catheter including a pressure sensor disposed within an annular wall of the catheter body such that an exterior surface of the pressure sensor and an outer surface of the catheter body are substantially aligned to form a smooth outer surface for a distal portion adjacent the pressure sensor;
advancing the sensing catheter over the guidewire to position the pressure sensor in the area of interest;
sensing fluid pressure in the area of interest;
withdrawing the pressure sensing catheter while leaving the guidewire in place; and
advancing a treating catheter over the guidewire to the area of interest.

18. The method of claim 17, wherein the sensing catheter includes a centering assembly disposed adjacent the pressure sensor and further including the step of deploying the centering assembly to engage the vessel walls in the area of interest and maintain the position of the catheter in the vessel prior to the step of sensing fluid pressure in the area of interest.
